(19) United States
(12) Patent Application Publication
(10) Pub. No.: US 2016/0230747 A1
(43) Pub. Date: Aug. 11, 2016

(54) LOCOMOTIVE SOLID-STATE DEVICE AND IMPLANTABLE MEDICAL SYSTEM HAVING THE SAME

(71) Applicant: NATIONAL TAIWAN UNIVERSITY, Taipei (TW)

(72) Inventors: SHEY-SHI LU, Taipei (TW); HUNG-WEI CHIU, Taipei City (TW); JIAN-YU HSIEH, Taipei (TW); YI-CHUN HUANG, Taipei (TW); PO-HUNG KUO, Taipei (TW); YAO-JOE YANG, Taipei (TW); LI-SHENG HSU, Taipei (TW)

(21) Appl. No.: 14/616,599

(22) Filed: Feb. 6, 2015

(51) Int. Cl. F03G 7/06; A61B 19/00; A61M 37/00; A61B 5/00

(52) U.S. Cl. CPC F03G 7/06; A61B 5/6846; A61B 19/00; A61M 37/00; A61B 2019/48; A61B 2560/0214

(57) ABSTRACT

The invention provides a locomotive solid-state device and an implantable medical system having the same. The locomotive solid-state device comprises a substrate, a signal receiving unit and a micro-controller. On the substrate at least one electrode is formed, and the signal receiving unit receives a plurality of electrical signals. The micro-controller electrically controls the electrode, in which the voltage applied on each electrode according to the electrical signals received by the signal receiving unit, so that the locomotive solid-state device moves along at least one direction.

### FIELD OF THE INVENTION

The present invention generally relates to a locomotive solid-state device and an implantable medical system having the same, and more particularly, certain embodiments of the invention relate to a locomotive solid-state device and an implantable medical system having the same that implanted inside a human body.

### BACKGROUND OF THE INVENTION

Implantable medical systems can be used in a human body, especially in some specific organs such as bladder, stomach or blood vessels to assist in diagnosing, bio-sensing, drug delivery, surgical operation, and so on. However, most of known implantable medical systems require external mechanism or tools for positioning in place before they operate, and the external mechanism or tools are sometimes structurally complicated or space wasted. For example, some of the implantable medical systems are able to be moved through magnetic force, so to achieve this, the human implanted with such an implantable medical system needs to stay on a specific magnetic bed with coil configured therein to build a great magnetic field. Sadly, the movement of the implantable medical system is limited in the magnetic direction. Therefore, there is needed to develop implantable medical systems which is locomotive to move in place.

### SUMMARY OF THE INVENTION

One aspect of the present invention is to provide a locomotive solid-state device and an implantable medical system having the same. According to one embodiment of the invention, the locomotive solid-state device is configured with at least one electrode among which the applied voltage is controlled by a micro-controller according to the electrical signals received by a signal receiving unit to move along at least one direction.

In another aspect of the invention, an embodiment of the invention is provided that a locomotive solid-state device comprises a signal receiving unit and a micro-controller. The signal receiving unit receives a plurality of electrical signals. The micro-controller, electrically coupled with at least one electrode and the signal receiving unit, controls the voltage applied on each electrode, which is greater enough to heat or electrolysis a mediate fluid, according to the electrical signals received by the signal receiving unit, so that the locomotive solid-state device moves along at least one direction.

In another aspect of the invention, an embodiment of the invention is provided that a locomotive solid-state device comprises a substrate, a signal receiving unit and a micro-controller. On the substrate at least one electrode is formed, and the signal receiving unit receives a plurality of electrical signals. The micro-controller is electrically coupled with the electrode and the signal receiving unit for controlling the voltage applied on each electrode according to the electrical signals received by the signal receiving unit, so that the locomotive solid-state device moves along at least one direction.

In at least one of the embodiment of the invention, the voltage applied on the electrode changes the state of a mediate fluid to apply a force/counterforce on the locomotive solid-state device, and thus it moves. Specifically, the voltage may be great enough to heat or electrolysis a mediate fluid, and the expanding mediate fluid or the gas generated by electrolyzing the mediate fluid apply a force/counterforce on the locomotive solid-state device. Multiple electrodes may be formed therein to add freedom of mobility, and preferably, the locomotive solid-state device can move forward, backward, leftward and rightward, or turn. To achieve this, for example, four and in a number of a multiple of four electrodes may be formed on the four sides and/or four corners of the substrate. The signal receiving unit may received the electrical signals through wire/wireless communication, and for the later case, the signal receiving unit may further comprise at least one signal coupling element, such as at least one coil, for coupling a plurality of wireless signals to generate the electrical signals. Moreover, the signal receiving unit may further comprise a power receiving element for receiving a wireless power carried by a plurality of electromagnetic waves. The locomotive solid-state device may comprise more elements or unit to handle the data processing, functional operation, power management, and so on, for example, a power management unit for managing the wireless power, a data demodulator for receiving and demodulating the electrical signals received by the signal receiving unit, a clock generator electrically coupled with the micro-controller for providing the micro-controller a clock signal, an electrode driving unit electrically coupled with the micro-controller to operate according to the command of the micro-controller for controlling the voltage applied on each electrode. Further, more structural elements or unit may be formed in the locomotive solid-state device, for example, a cover encompassing the electrode may be mounted therein to form a cavity, a guiding channel to guide the movement may be positioned adjacent to the electrode, and so on. All the elements and units of the locomotive solid-state device may be positioned on the substrate to implement the locomotive solid-state device in a system-on-chip manner. However, please noted that the invention is not limited to these specific examples.

In another aspect of the invention, an embodiment of the invention is provided that an implantable medical system comprises any of aforesaid examples of the locomotive solid-state devices and an actuating module, mounting on the locomotive solid-state device to operate at least one medical, diagnostic and/or bio-sensitive function.

In at least one of the embodiment of the invention, the actuating module comprises at least one device providing medical, diagnostic, bio-sensitive function(s), for example but not limited to a biosensor, drug delivery device, camera device, operative instrument, etc. The implantable medical system may comprise more elements or unit to handle the data processing, power management, and so on, for example, a wireless communication device for communicating with the locomotive solid-state device to control the operation and the movement of the locomotive solid-state device.

### BRIEF DESCRIPTION OF THE DRAWINGS

Exemplary embodiments will be more readily understood from the following detailed description when read in conjunction with the appended drawing, in which:

FIG. 1, which shows a scenario diagram of a implantable medical system of an exemplary embodiment according to the invention;

FIG. 2 shows a block diagram of a locomotive solid-state device of an exemplary embodiment according to the invention;

FIG. 3 a perspective view of a locomotive solid-state device of an exemplary embodiment according to the invention;

FIG. 4 a perspective view of a locomotive solid-state device of an exemplary embodiment according to the invention;

FIG. 5 shows a perspective view of an electrode of an exemplary embodiment according to the invention;

FIG. 6 shows a perspective view of an electrode of an exemplary embodiment according to the invention;

FIG. 7 shows a schematic diagram of a regulator of an exemplary embodiment according to the invention;

FIG. 8 shows a schematic diagram of a data demodulator of an exemplary embodiment according to the invention;

FIG. 9 shows a schematic diagram of a clock generator of an exemplary embodiment according to the invention;

FIG. 10 shows a schematic diagram of a power-on reset unit of an exemplary embodiment according to the invention;

FIG. 11 shows a schematic diagram of a micro-controller of an exemplary embodiment according to the invention;

FIG. 12 shows a example of the signals transmitted by a micro-controller of an exemplary embodiment according to the invention;

FIG. 13 shows a schematic diagram of a electrode driving unit of an exemplary embodiment according to the invention;

FIG. 14 shows a perspective view of a locomotive solid-state device of an exemplary embodiment according to the invention; and

FIG. 15(a)˜15(c) shows a cross-section view of a locomotive solid-state device of an exemplary embodiment according to the invention.

### DESCRIPTION OF EMBODIMENTS OF THE INVENTION

For a more complete understanding of the present disclosure and its advantages, reference is now made to the following description taken in conjunction with the accompanying drawings, in which like reference numbers indicate like features. Persons having ordinary skill in the art will understand other varieties for implementing example embodiments, including those described herein. The drawings are not limited to specific scale and similar reference numbers are used for representing similar elements. As used in the disclosures and the appended claims, the terms “example embodiment,” “exemplary embodiment,” and “present embodiment” do not necessarily refer to a single embodiment, although it may, and various example embodiments may be readily combined and interchanged, without departing from the scope or spirit of the present invention. Furthermore, the terminology as used herein is for the purpose of describing example embodiments only and is not intended to be a limitation of the invention. In this respect, as used herein, the term “in” may include “in” and “on”, and the terms “a”, “an” and “the” may include singular and plural references. Furthermore, as used herein, the term “by” may also mean “from”, depending on the context. Furthermore, as used herein, the term “if” may also mean “when” or “upon”, depending on the context. Furthermore, as used herein, the words “and/or” may refer to and encompass any and all possible combinations of one or more of the associated listed items.

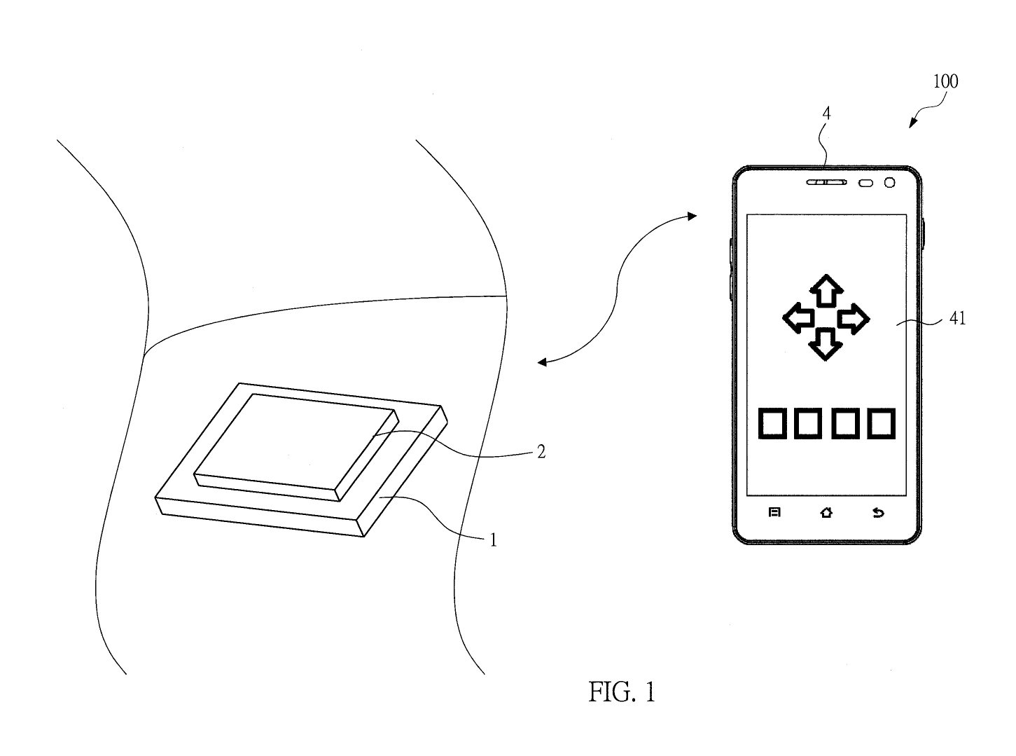

Please refer to FIG. 1, which shows a scenario diagram of a implantable medical system 100 of an exemplary embodiment according to the invention. The implantable medical system 100 comprises a locomotive solid-state device 1, an actuating module 2 and a wireless communication device 4, such as a mobile phone. The exemplary locomotive solid-state device 1 may be configured with the actuating module 2 as a separated device to be implanted in a part of a human body, such as some specific organs like a bladder, stomach or blood vessels assist in diagnosing, bio-sensing, drug delivery, surgical operation, and so on. The locomotive solid-state device 1 preferably bears the actuating module 2 and allows the actuating module 2 floating on a surface of a mediate fluid, which may be a kind of body fluid inside the part of the human body or some predetermined liquid or gas, or diving in the mediate fluid. Here, the locomotive solid-state device 1 serves as a resource of the capability of movement, the actuating module 2 mounted on the locomotive solid-state device 1 operates to provide medical, diagnostic and/or bio-sensitive function(s), for example but not limited to a biosensor, drug delivery device, camera device, capsule endoscope, operative instrument, etc., and the wireless communication device 4 provides an interface 41 to communicate with the locomotive solid-state device 1 to control the operation and the movement of the locomotive solid-state device 1. Through the interface 41, users may control the movement of the locomotive solid-state device 1, acknowledge the position of the locomotive solid-state device 1 or the feedback information of the actuating module 2, and operates the actuating module 2 in action when the a implantable medical system 100 is in place. Please noted that the wireless communication device 4 may be replaced by wire-communication device providing an interface in another embodiment of the invention.

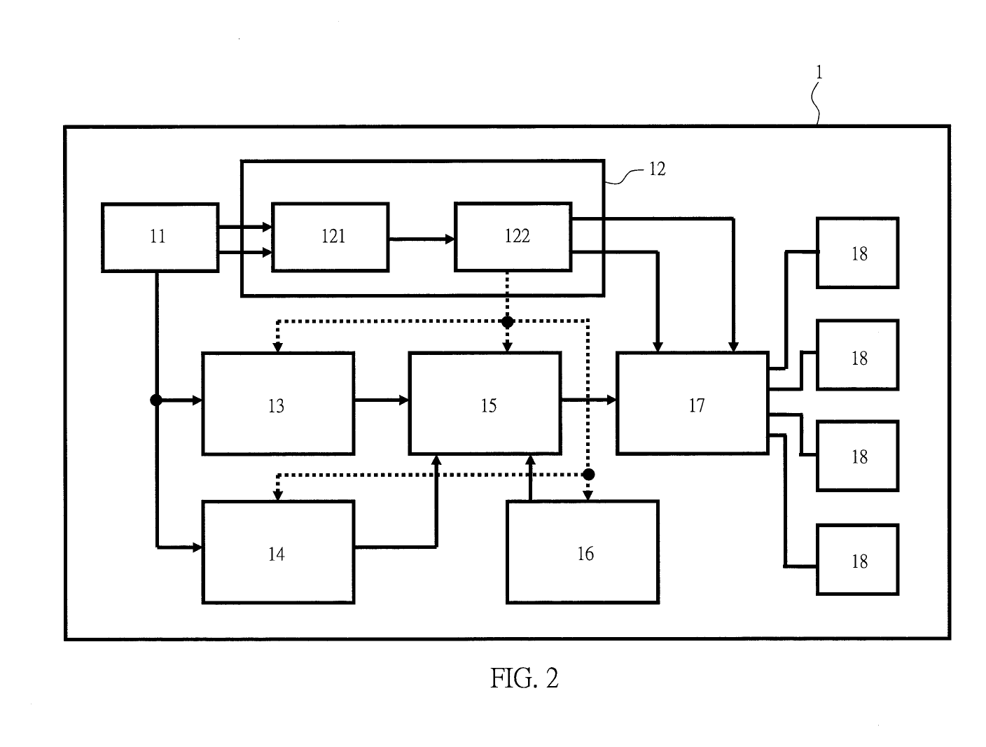

To achieve the mobility, at least one electrode may be implemented, and multiple electrodes may be formed therein to add the freedom of mobility. The material of the electrode(s) may be chosen from but not limited to the group comprising of Au and Ti/Pt alloy, which performs good biological compatibility. Please refer to FIG. 2, which shows a schematic diagram of a locomotive solid-state device of an exemplary embodiment according to the invention. The exemplary locomotive solid-state device 1 comprises a signal receiving unit 11, a power management unit 12, a data demodulator 13, a clock generator 14, a micro-controller 15, a power-on reset unit 16, an electrode driving unit 17 and four electrodes 18. Please noted that the number of the electrodes is for example. Here, all the elements and units of the locomotive solid-state device 1 is exemplarily but not limited to positioned on one single substrate to implement the locomotive solid-state device in a system-on-chip manner to condense the size of the whole system, and in some embodiment(s) of the invention, the elements and units of the locomotive solid-state device 1 may be configured in several substrates which are fixedly attached with each other in a multi-chip module way. The signal receiving unit 11 receives a plurality of electrical signals from an external device, such as the wireless communication device 4 shown in FIG. 1 with wire/wirelessly and moreover a wireless power carried by a plurality of electromagnetic waves. Here, the signal receiving unit 11 is constructed with two on-chip coils (not shown), and one serves as a signal coupling element for coupling a plurality of wireless signals to generate the electrical signals and the other serves as a power receiving element to receive the wireless power.

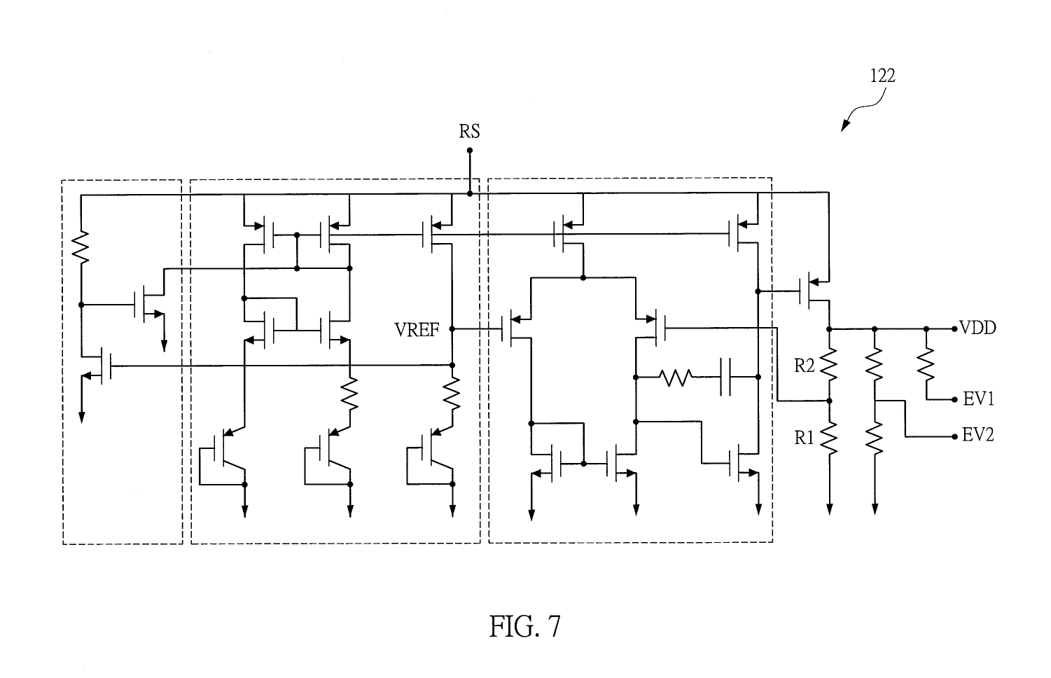

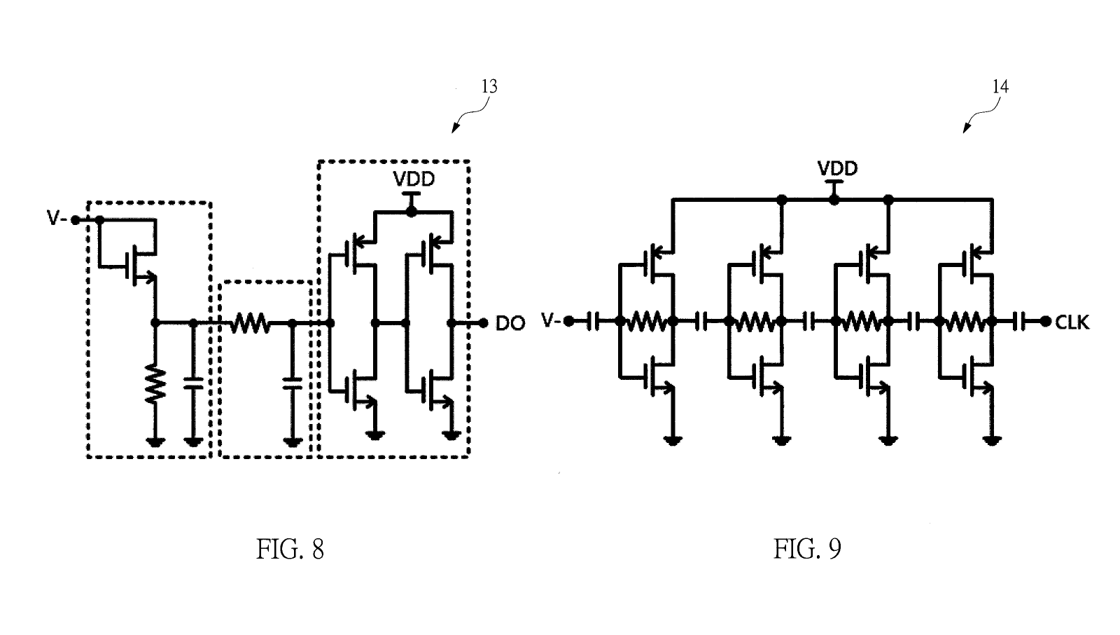

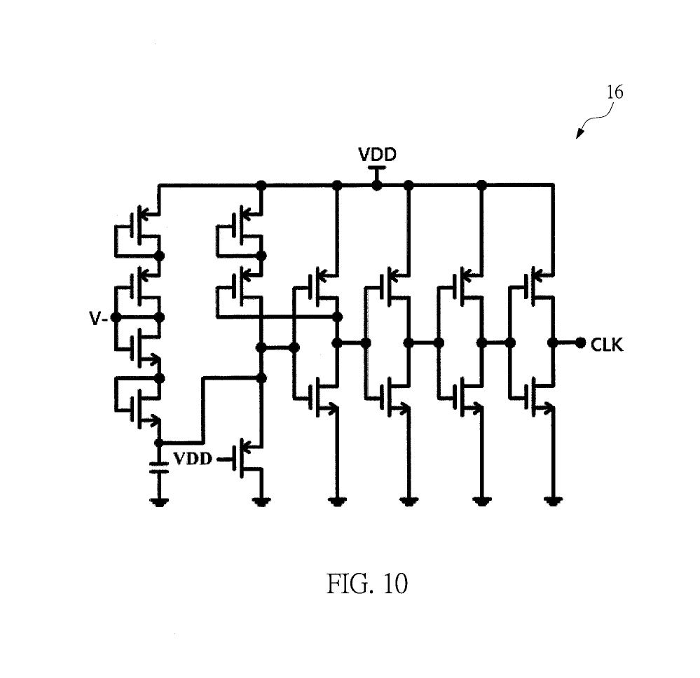

The micro-controller 15 is electrically coupled with the signal receiving unit 11 with the intermediary data demodulator 13 and the clock generator 14, electrically coupled with the electrodes 18 with the intermediary electrode driving unit 17, and electrically coupled with the power-on reset unit 16. The power management unit 12 assists in managing the wireless power first with a power rectifier 121 and then a regulator 122. Please see an exemplary schematic diagram of the regulator 122 shown in FIG. 7. The data demodulator 13 demodulates the electrical signals received by the signal receiving unit 11 before sending to the micro-controller 15. Please see an exemplary schematic diagram of the data demodulator 13 shown in FIG. 8. The clock generator 14 electrically coupled with the micro-controller 15 provides the micro-controller 15 a clock signal for operation. Please see an exemplary schematic diagram of the clock generator 14 shown in FIG. 9. The power-on reset unit 16 generates a power-on signal of POR after the power have stabilized at an appropriate level. Please see an exemplary schematic diagram of the power-on reset unit 16 shown in FIG. 10. Please noted that the power management unit 12, the data demodulator 13, the clock generator 14 and the power-on reset unit 16 are configured for data processing, functional operation or power management here, and in some other embodiment(s) of the invention, a different structure may be constructed for these purposes.

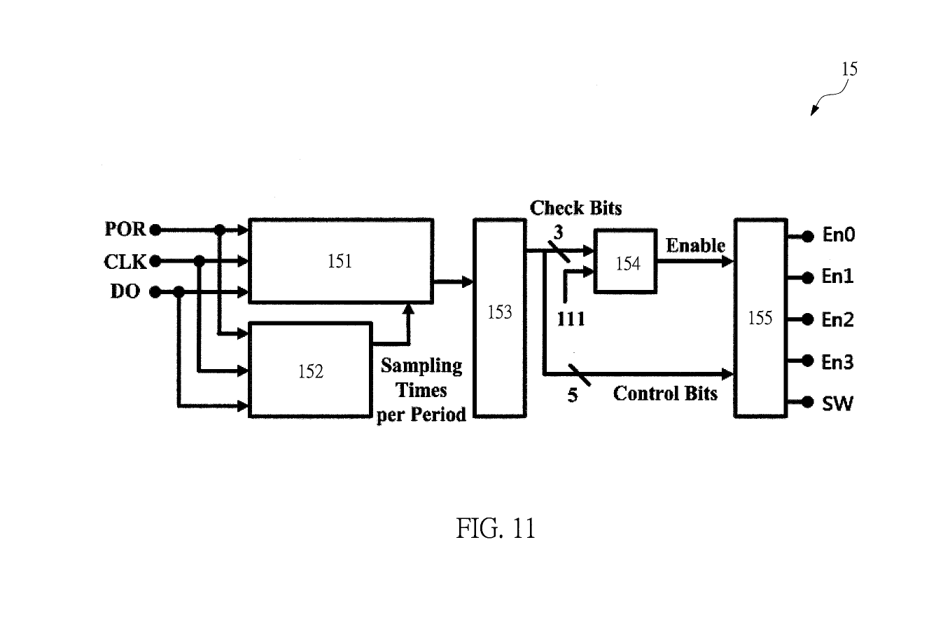

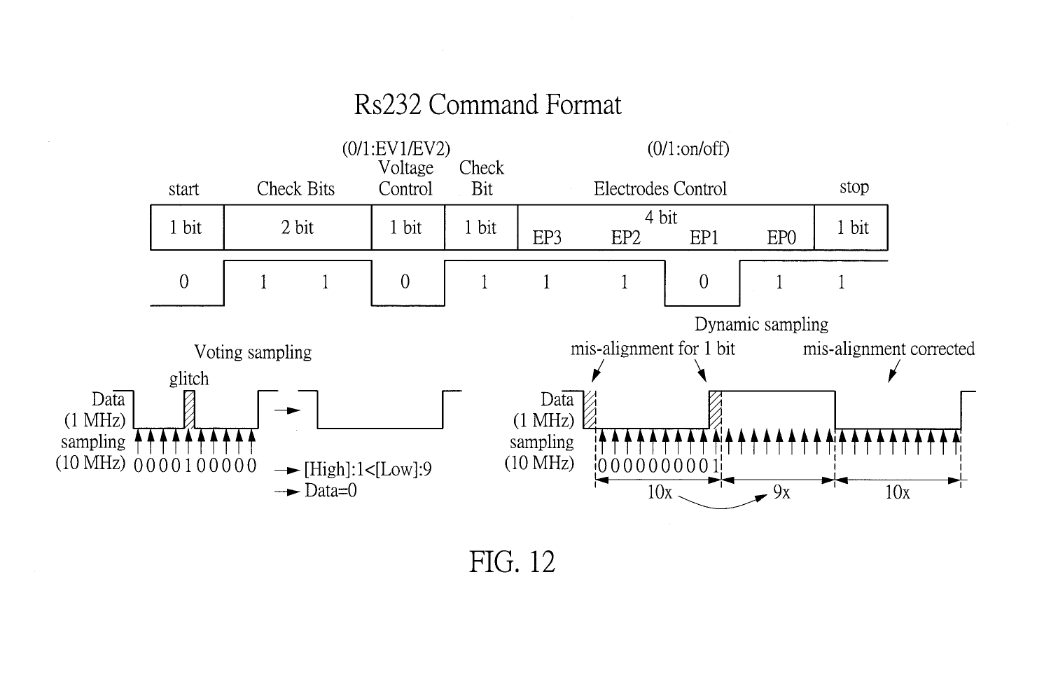

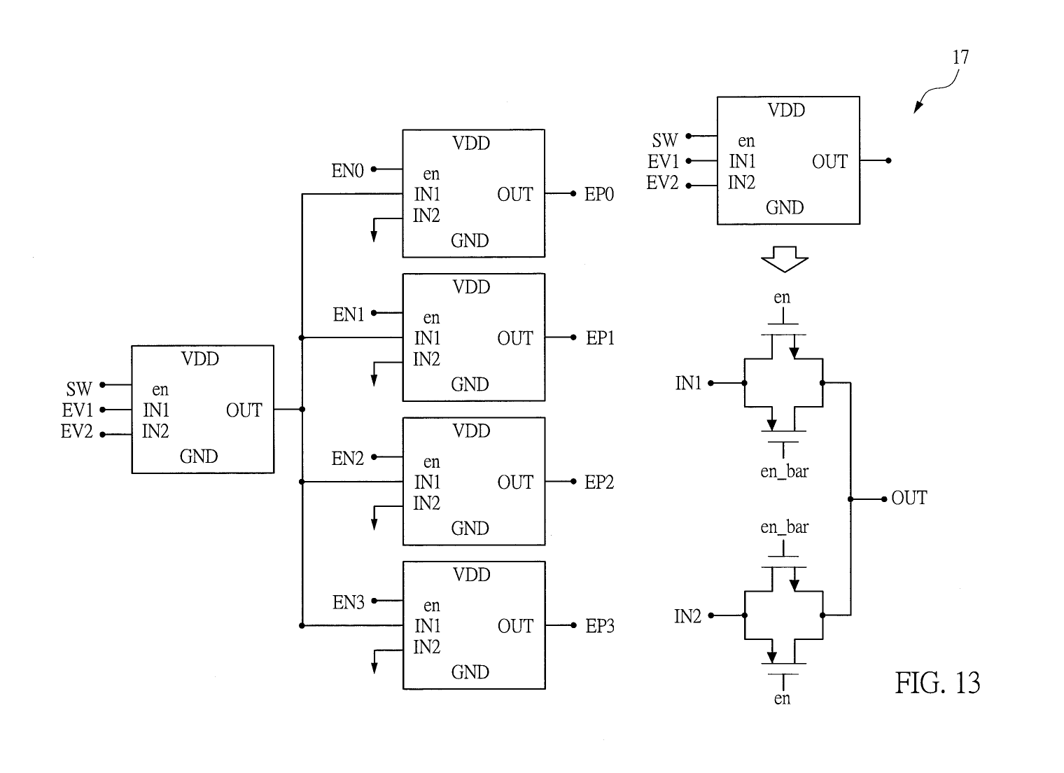

The micro-controller 15 controls the voltage applied on each electrode 18 according to the electrical signals received by the signal receiving unit 11, preferably, the voltage applied on the electrode 18 changes the state of the mediate fluid to apply a force/counterforce on the locomotive solid-state device 1, and thus it moves along at least one direction. Please see an exemplary schematic diagram of the micro-controller 15 shown in FIG. 11. The micro-controller 15 comprises a voting sampling element 151, dynamic sampling element 152, UART receiver 153, logic controller 154 and a decoder 155. By using the dynamic sampling, the misalignment of received data DO could be corrected. Also, by using voting sampling, the correctness of received data DO could be decided in majority by voting. After sampling, the sampled data will be packed in format of RS-232 by UART receiver. Here, the micro-controller 15 transmits signals in the form of RS232 command format, which is shown in FIG. 12, exemplarily. Four bits, EP0˜EP3 in the signals transmitted by the micro-controller 15 are used to control the voltage of the four electrodes by the electrode driving unit 17, which comprises a set of analog switches here. Please see an exemplary schematic diagram of the electrode driving unit 17 shown in FIG. 13. The voltage applied on the electrode 18 may be great enough to heat or electrolysis the mediate fluid. In the previous case, the heated mediate fluid expands, in the later case, the electrolyzed mediate fluid generates gas, and both apply a force/counterforce on the locomotive solid-state device 1, so the locomotive solid-state device 1 moves as a result.

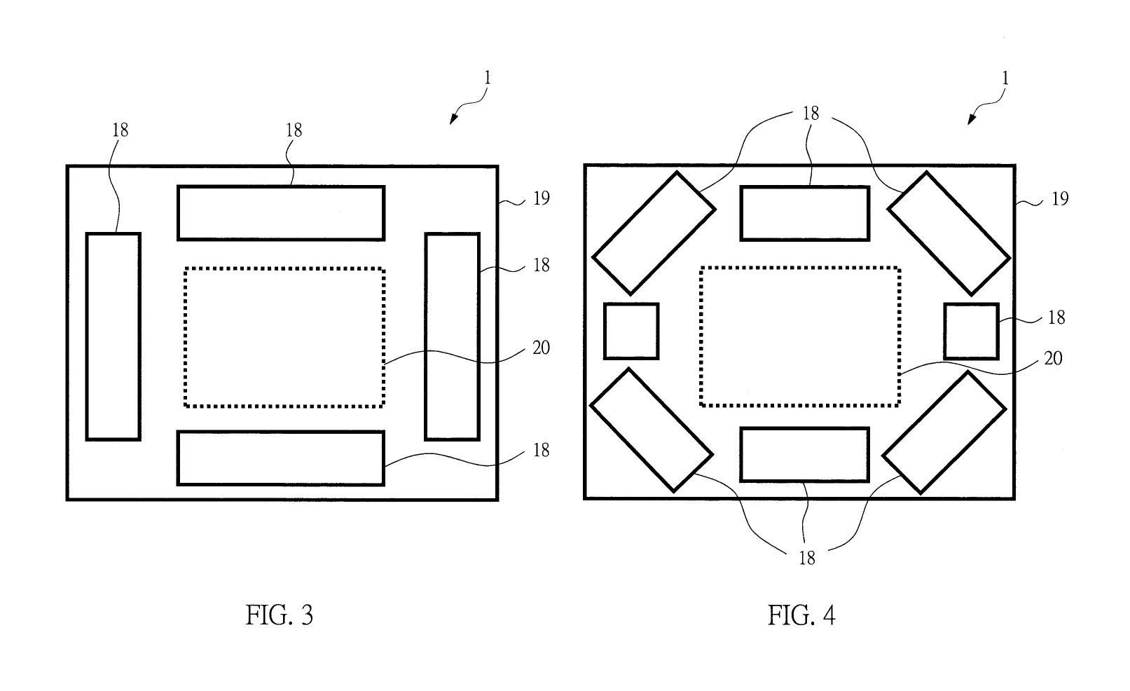

The electrodes 18 may be formed on a substrate. Please refer to FIG. 3, which shows a perspective view of a locomotive solid-state device 1 of an exemplary embodiment according to the invention. Here, the number of the electrodes 18 is exemplarily 4, a multiple of four positioned at the four sides of the substrate, and the other units or some of the other units in the locomotive solid-state device 1, such as signal receiving unit, a power management unit, a data demodulator, a clock generator, a micro-controller, a power-on reset unit and/or an electrode driving unit may be formed or electrically contacted in the area 20. For example, when the micro-controller 15 controls to apply a high voltage or a relatively high voltage on the top electrode 18 shown in FIG. 3, which is high enough to electrolysis the mediate fluid, bubbles or more bubbles generated under the electrode, and than the locomotive solid-state device 1 move forward. In a similar way, the locomotive solid-state device 1 may move backward, leftward and rightward.

Please refer to FIG. 4, which shows a perspective view of a locomotive solid-state device 1 of an exemplary embodiment according to the invention. Here, the number of the electrodes 18 is exemplarily 4, a multiple of four positioned at the four corners of the substrate, and the other units or some of the other units in the locomotive solid-state device 1, such as signal receiving unit, a power management unit, a data demodulator, a clock generator, a micro-controller, a power-on reset unit and/or an electrode driving unit may be formed or electrically contacted in the area 20. For example, when the micro-controller 15 controls to apply a high voltage or a relatively high voltage on the top electrode 18 and then the top left electrode 18 shown in FIG. 4, which is high enough to electrolysis the mediate fluid, bubbles or more bubbles generated under the electrode, and than the locomotive solid-state device 1 move forward and then turn right. In a similar way, the locomotive solid-state device 1 may move backward, leftward and rightward and turn about different directions.

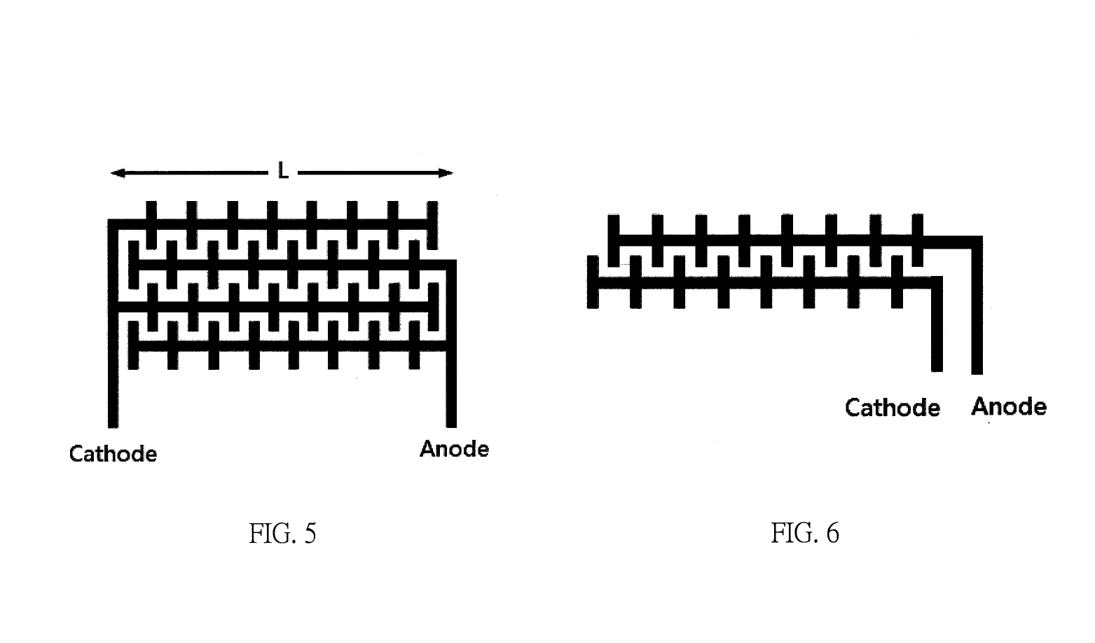

Please refer to FIG. 5, which shows a perspective view of an electrode of an exemplary embodiment according to the invention. Two strands of cathode and two strands of anode form alternately pattern on the substrate. One more exemplary embodiment for the electrode is shown in FIG. 6. Please noted that other types or forms of pattern for the electrode may be used in the invention.

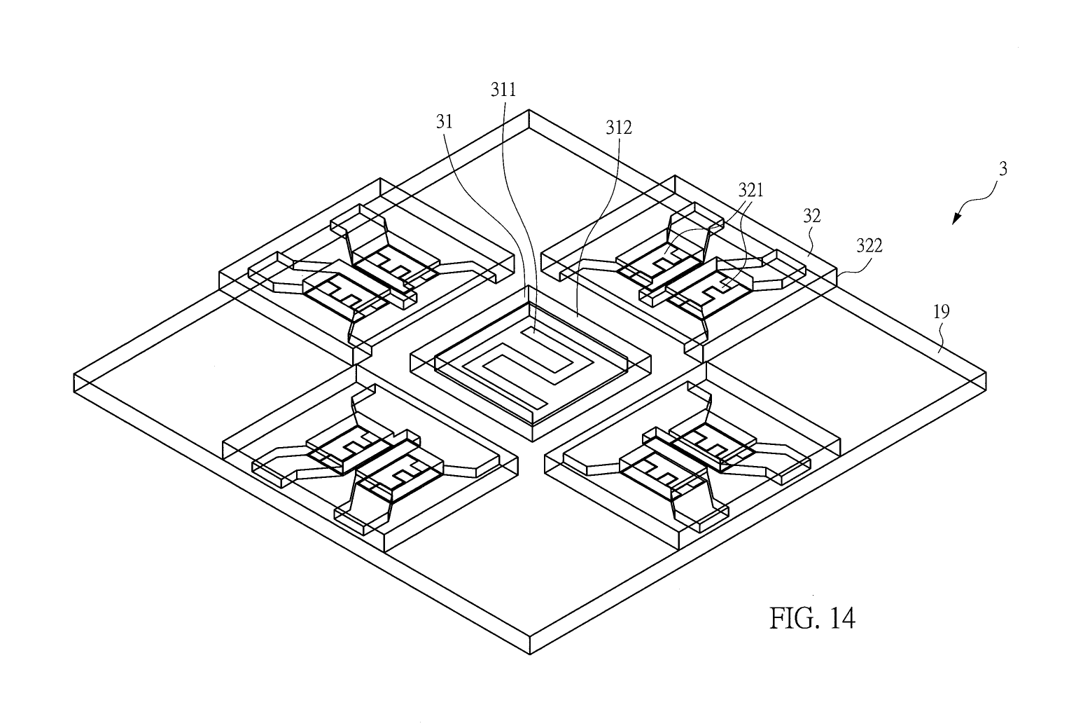

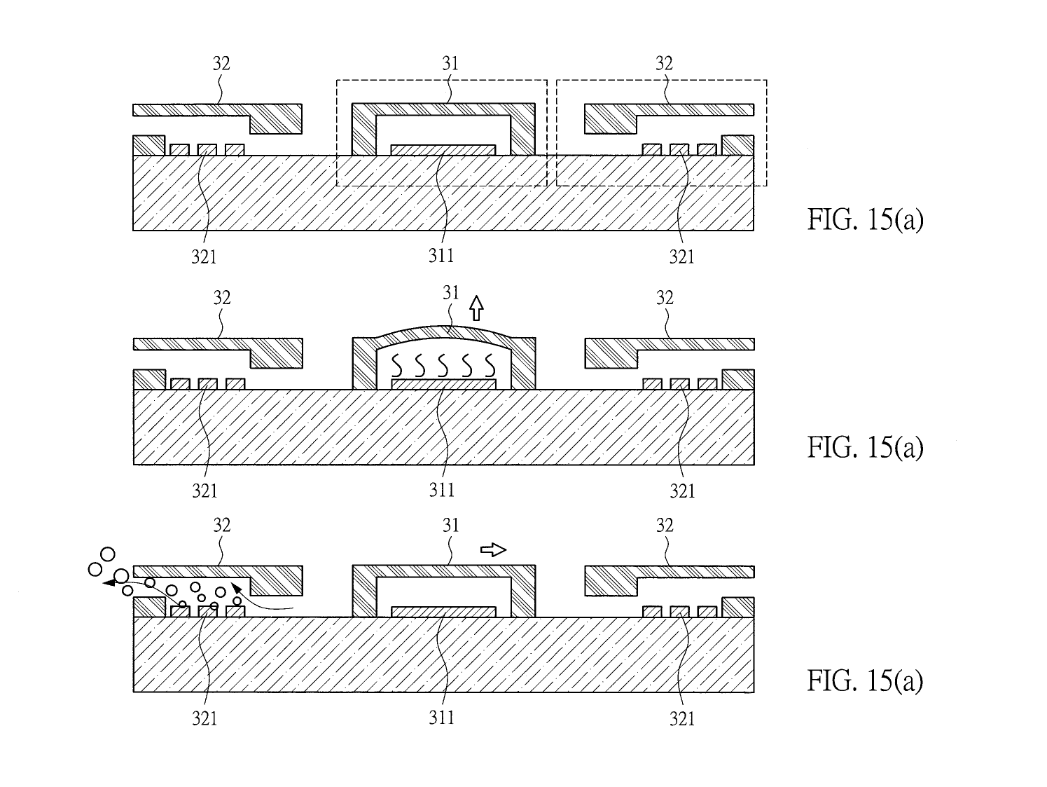

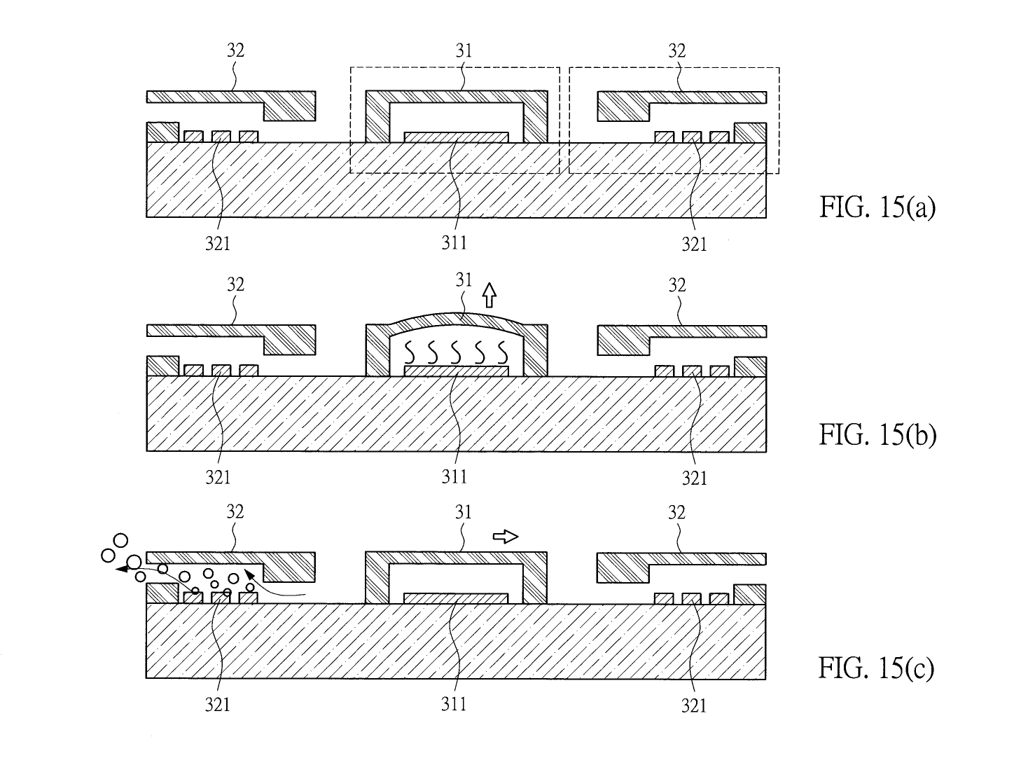

Please refer to FIG. 14 and FIG. 15(a), FIG. 14 shows a perspective view of a locomotive solid-state device 3 of an exemplary embodiment according to the invention, and FIG. 15(a) shows a cross-section view of a locomotive solid-state device 3 of an exemplary embodiment according to the invention. A cavity 31 is formed by a cover 312 encompassing the electrode 311 in the center of the substrate for adjusting the buoyancy by controlling the voltage applied on electrode 311. As shown in FIG. 15(b), when a high voltage is applied, a mediate fluid in the cavity, here is a gas for example, is heated for expansion, the buoyancy changes, and then the locomotive solid-state device 3 moves upward as a result. Similarly, when a low voltage is applied, the locomotive solid-state device 3 moves downward. Four guiding channels 32 adjacent to the electrodes 321 are formed at the four sides of the substrate 19 with sidewalls 322. The guiding channels 32 may be used for control the movement of the locomotive solid-state device 3. For example, as shown in FIG. 15(c), when a high voltage is applied, a mediate fluid in the guiding channel, here is a liquid for example, is electrolyzed to generate bubbles, and then the locomotive solid-state device 3 moves rightward. Similarly, the locomotive solid-state device 3 may move forward, backward and leftward with properly controlling the voltage applied on the electrodes 321.

It is to be understood that these embodiments are not meant as limitations of the invention but merely exemplary descriptions of the invention with regard to certain specific embodiments. Indeed, different adaptations may be apparent to those skilled in the art without departing from the scope of the annexed claims. For instance, it is possible to add bus buffers on a specific data bus if it is necessary. Moreover, it is still possible to have a plurality of bus buffers cascaded in series.

What is claimed is:

1. A locomotive solid-state device, comprising:
a substrate, on which at least one electrode is formed;
a signal receiving unit, receiving a plurality of electrical signals; and
a micro-controller, electrically coupled with the electrode and the signal receiving unit for controlling the voltage applied on each electrode according to the electrical signals received by the signal receiving unit, so that the locomotive solid-state device moves along at least one direction.

2. The locomotive solid-state device according to claim 1, further comprising:
at least one another electrode formed on the substrate.

3. The locomotive solid-state device according to claim 2, wherein the number of the electrodes is a multiple of four and the micro-controller controls the voltage applied on each electrode according to the electrical signals received by the signal receiving unit, so that the locomotive solid-state device moves forward, backward, leftward and rightward.

4. The locomotive solid-state device according to claim 2, wherein the number of the electrodes is a multiple of four and positioned at the four sides of the substrate.

5. The locomotive solid-state device according to claim 2, wherein the number of the electrodes is a multiple of four and positioned at the four corners of the substrate.

6. The locomotive solid-state device according to claim 1, wherein the signal receiving unit further comprising at least one signal coupling element for coupling a plurality of wireless signals to generate the electrical signals.

7. The locomotive solid-state device according to claim 6, wherein the signal coupling element comprises at least one coil.

8. The locomotive solid-state device according to claim 1, wherein the signal receiving unit further comprising a power receiving element for receiving a wireless power carried by a plurality of electromagnetic waves.

9. The locomotive solid-state device according to claim 8, further comprising:
a power management unit, managing the wireless power;
a data demodulator, receiving and demodulating the electrical signals received by the signal receiving unit; and
a clock generator, electrically coupled with the micro-controller to provide the micro-controller a clock signal.

10. The locomotive solid-state device according to claim 1, further comprising:
an electrode driving unit, electrically coupled with the micro-controller to operate according to the command of the micro-controller for controlling the voltage applied on each electrode.

11. The locomotive solid-state device according to claim 1, wherein the voltage applied on each electrode is greater enough to electrolysis a mediate fluid.

12. The locomotive solid-state device according to claim 1, wherein the voltage applied on each electrode is greater enough to heat a mediate fluid.

13. The locomotive solid-state device according to claim 1, wherein the signal receiving unit and the micro-controller are both formed on the substrate.

14. The locomotive solid-state device according to claim 1, further comprising a cover encompassing the electrode to form a cavity.

15. The locomotive solid-state device according to claim 1, further comprising a guiding channel positioned adjacent to the electrode to guide the movement.

16. A locomotive solid-state device, comprising:
a signal receiving unit, receiving a plurality of electrical signals;
a micro-controller, electrically coupled with at least one electrode and the signal receiving unit for controlling the voltage applied on each electrode, which is greater enough to heat or electrolysis a mediate fluid, according to the electrical signals received by the signal receiving unit, so that the locomotive solid-state device moves along at least one direction.

17. The locomotive solid-state device according to claim 16, wherein the signal receiving unit further comprising at least one signal coupling element for coupling a plurality of wireless signals to generate the electrical signals.

18. A implantable medical system, comprising:
a locomotive solid-state device as claimed in claim 1; and
an actuating module, mounting on the locomotive solid-state device to operate at least one medical, diagnostic or bio-sensitive function.

19. The implantable medical system according to claim 18, wherein the actuating module comprises at least one of a biosensor, drug delivery device, camera device or operative instrument.

20. The implantable medical system according to claim 18, further comprising a wireless communication device, communicating with the locomotive solid-state device to control the operation and the movement of the locomotive solid-state device.
